(19) United States
(12) Patent Application Publication
(10) Pub. No.: US 2025/0344988 A1
(43) Pub. Date: Nov. 13, 2025

(54) Method and Apparatus for Wearable Device with Timing Synchronized Interface for Cognitive Testing

(71) Applicant: SENS.AI INC, Whistler (CA)

(72) Inventors: Paola Telfer, Whistler (CA); Corey Julihn, Langley (CA)

(21) Appl. No.: 18/869,273

(22) PCT Filed: May 25, 2023
(86) PCT No.: PCT/IB2023/055392

§ 371 Date: Nov. 25, 2024

Related U.S. Application Data
(60) Provisional application No. 63/345,880, filed on May 25, 2022.

(51) Int. Cl. A61B 5/378; A61B 5/00; A61B 5/374; A61B 5/38

(52) U.S. Cl. CPC A61B 5/378; A61B 5/0006; A61B 5/374; A61B 5/38

(57) ABSTRACT

Devices and methods for analyzing and monitoring electroencephalographic (EEG) electrical activity evoked during neurocognitive tests using event-related potential (ERP) and evoked and induced EEG oscillations (ERO) measures in human users. Electroencephalography (EEG) devices in the form of wearable apparatus with headphones and cognitive test interface with concurrent EEG monitoring and evaluation of electrical activity generated by a person's brain during stimulation are described, along with description of methods for testing person's cognitive and physiological state during cognitive tests using the provided devices. EEG sensors for detecting EEG responses during cognitive tests using event-related potentials (ERP) and evoked and induced EEG oscillations (ERO) for evaluation of user's cognitive status and functional outcomes of treatment. Additionally, devices and sensors for monitoring heart rate, heart rate variability (HRV), electrocardiogram (EKG), and photoplethysmography (PPG), and analysis of evoked heart rate, HRV, and pulse volume responses during cognitive tests. The devices may be used to assess psychophysiological responses and assist users with monitoring mental performance, cognitive function, stress, anxiety, fatigue, mood, behavioral performance and mental focus and acuity.

### PRIOR RELATED APPLICATIONS

This application claims the benefit of priority of prior-filed U.S. Provisional Application 63/345,880, filed May 25, 2022.

### FIELD OF THE INVENTION

The present invention relates to devices and methods for stimulating, monitoring, and analyzing electrical activity generated by the brain of a person. Specifically, the invention provides electroencephalography (EEG), electrocardiogram (EKG), and photoplethysmography (PPG) devices and interface for monitoring and stimulating electrical activity generated by a person's brain and heart during cognitive tests. The present invention also relates to devices used to execute cognitive tests including sensors, visual display units and input trigger units. Further the present invention relates to methods for assessment of electrocortical responses using EEG and event-related potential (ERP) responses and event-related EEG oscillations (ERO) evoked during cognitive function tests.

### BACKGROUND OF THE INVENTION

Event-related potential (ERP) recorded in cognitive tests is one of the most informative methods of exploration and monitoring of the stages of information processing in the brain. Measures such as amplitude and latency of selected ERP waves recorded at specific topographies allow analysis of sensory and perception-relation processes, as well as higher-level processing stages including attention, cortical inhibition, memory update, error monitoring and other cognitive activities termed also under executive functions definition (Luck, 2014). ERPs provide a method of investigation of cognitive processes not only in typical individuals, but also provides a sensitive tool to assess differences in patients with neurological and psychiatric conditions and to monitor treatment outcomes. Despite significant advances in functional neuroimaging such as fMRI, ERP-based metrics still represents an important instrument in neurology and psychiatry, since some neuropsychiatric diseases correlate with known alterations in ERP patterns that can serve as valid biological neuromarkers for functional diagnostic or for better understanding of the disturbed cognitive functions in psychiatric and neurological conditions.

During the normal process of aging, humans may experience a certain amount of age-related cognitive decline (ARCD) resulting in increased difficulty in demanding situations and decreased ability to focus attention under time pressure conditions. Individuals with ARCD experience decline in cognitive functioning resulting in decrement of performance effectiveness in tasks that require attention, short-and long-term memory, fast motor reaction, speeded decision making, as well as processing and comprehension of situational demands. Tests using ERP and EEG oscillations (ERO) are known to be one of the best techniques to evaluate the status of cognitive decline both in elderly and in predisposed younger users or those after diseases or disorders known to be associated with decreased cognitive functioning, such as concussion, traumatic brain injury (TBI), or infectious diseases resulting in post-disease “brain fog”.

Other useful EEG measures in cognitive tests are based on wavelet-based time-frequency analysis of EEG oscillations (ERO) in response to stimuli in cognitive tests. More information about time-frequency wavelet-based analysis of EEG and about EEG evoked and induced gamma oscillations (ERO) can be found in the publication of Tallon-Baudry & Bertrand (1999) that describes evoked and induced EEG gamma oscillations (30-100 Hz with most usable gamma range being in 35-45 Hz range). The review focuses on the literature on gamma oscillatory activities in humans and describes the different types of gamma responses and how to analyze them. Evidence presented by researchers suggests that one particular type of gamma activity, specifically induced gamma oscillations can be observed during the construction of an object representation is discussed. The paper has illustrations of evoked and induced gamma EEG oscillations and explanation of their role.

Neuronal gamma-band oscillations, along with other EEG bands oscillations, can be recorded at different scalp topographies (as well as in cortical and subcortical areas), and can be evoked or induced by different stimuli or tasks, such as, for instance, ERP design tests. Event-related oscillatory activity (ERO) in various frequency bands (e.g., delta, theta, alpha, beta, gamma, etc.) reflects different aspects and stages of information processing. Alpha oscillatory responses increase with simple working memory tasks and decrease with demanding memory tasks. Beta oscillatory responses are important in attention related tasks and some affective tests, for instance recognition of facial expression in humans. Event-related theta oscillatory responses have been proposed to be related to the memory processes. Beta and gamma oscillations are considered to reflect higher order information processes.

Wavelet analysis is useful for single trial analysis of EEG oscillation in rare responses, such as for instant response-locked ERP occurring after committed error in speeded cognitive tasks requiring motor response. Clemans et al. (2012) reported that response-locked ERP used as measures of error processing are the error-related negativity (ERN) and the error-related positivity (Pe) that occur following committed error in speeded reaction time tests and can be recorded in a form of low frequency (4-8 Hz) EEG oscillations at the midline frontal and frontocentral EEG sites. Error processing using time-frequency analysis in the form of a wavelet transform is described as an alternative method to isolate a theta waveform in the time-frequency domain and to obtain a single time-frequency correlates of ERN and Pe for each error trial. These results of the study indicate that the suggested alternative single trial time-frequency error analysis method is suitable for detection of error-related processes both in healthy individuals and patients with psychiatric conditions.

Davoudi et al. (2021) describe frequency-amplitude coupling as a new approach for decoding of attention-related processes in cognitive tasks. The method has been described in said study as reflecting information processing in the brain and references cross-frequency coupling. It is generally assumed that some EEG frequencies demonstrate phase-amplitude coupling processes, for instance theta-gamma phase amplitude coupling plays a crucial role in perception, memory, and attention (Canolty et al., 2006; Koster et al., 2014).

There are numerous patents that describe apparatus and methods for wearable EEG systems for various indications and multiple embodiments, including those that disclose applications of various event-related potential (ERP) tests (U.S. Pat. No. 8,391,966B2, U.S. Pat. No. 8,938,301B2, U.S. Pat. No. 9,675,292B2, US 2012/0330178A1). Some of them have detailed description of stimulus-locked ERPs and response-locked error-related negativity (ERN) and error-related positivity (Pe) potentials (US 2021/0338140A1). There are patents (e.g., U.S. Pat. No. 8,731,650B2) describing application of time-frequency analysis of EEG responses using wavelet transformation and justifying usability of the method for single trial analysis of EEG responses. The literature has many publications on gamma oscillations in tasks similar or even the same as ERP paradigm, including description of evoked and induced gamma, and literature about theta and gamma and other EEG rhythms phase-amplitude coupling as useful measures of cognitive functions (Lisman & Jensen, 2013; Koster et al., 2014). Evoked and induced gamma oscillations, as well as theta oscillations, and their coupling is an area not covered in patents or publications related to wearable devices in cognitive tests. There are patents that list, as possible embodiments, the inclusion of other biometrics along with EEG, such as for instance heart rate (HR), heart rate variability (HRV) and other vitals signals. There are many patents and published scientific literature that focus on HRV biofeedback and EEG biofeedback and usability of biofeedback training for various clinical and performance improvement applications (Lehrer & Gevirtz, 2014; Sherlin et al., 2011).

There are patents filed that describe a device with EEG and event-related potential (ERP) functions, including those that have methods of ERP analysis disclosed and several cognitive tests with ERP recording described. There are among them wearable systems that can analyze and assess a person's brain health by integrating the use of EEG and ERP metrics during cognitive testing. Such systems are able to provide for early detection of neurological and psychiatric disorders such as mild cognitive impairment (MCI), dementia, including Alzheimer's disease, and other dementia-type disorders, as well as brain injury states such as mild traumatic brain injury (mTBI). Some of these patents, e.g., U.S. Pat. No. 9,675,292 B2 and EP 2260760 B1 by Fadem describe ERP systems suitable for clinical use that includes an integrated headset that performs an evoked response (ERP) test. Other patents, i.e., WO2020/223397 A1 by Mcloughlin, describe mental fitness assessment systems in healthy person and claim that EEG and ERP measures are indicative of an emotional or cognitive state of the person; and allow assessment of a mental fitness state of the person based on the electrocortical metrics.

These prior art devices and methods present multiple limitations which prevent their practical use by non-trained individuals, outside of clinical settings, without additional equipment, or are limited to auditory ERP analysis, and/or are not mobile. These limitations include only providing for auditory event stimuli, requiring a trained clinician to place and monitor the sensors and device, or requiring external computer systems. Moreover, due to the timing constraints of stimulus based cognitive testing, the required external computer systems are highly restrictive, often requiring a specified graphics card and monitor.

Stimulus based cognitive testing is extremely sensitive to timing requirements. Prior art systems typically utilize one device to capture biometric sensor data as well as behavioral responses from the user and a separate device to provide visual stimuli to the user. These systems can be considered non-time synchronized, wherein the timing uncertainty from the sensors and the timing uncertainty from the display of stimulus combine to create even greater timing uncertainty. As an example, these systems typically use displays with a refresh rate of 120 Hz resulting in a stimulus uncertainty of 8.33 ms which adds to any timing uncertainty from the sensor sample rate. As a result of this uncertaining in the timing of the onset of the stimuli, the data suffers from a timing jitter leading to a blurring of the analyzed ERP data and unreliable latency measures of ERP peaks. Prior art does include additional devices used to measure the display output in order to synchronize the visual stimulus timing with the sensor data. However, these solutions require additional hardware and wires, and further restrict the system to a stationary location.

ERP and other stimulus based cognitive tests are characterized by inherited delays associated with jitters related to EEG signal and or event marker transmission. Some delays are due to the delays of EEG processing and those related to the stimulus event mark delays due to stimulus signal marker stamp arrival. Certain delays are due to software processing of EEG signal and graphics. There is a need to synchronize stimulus event marker and EEG signal for accurate estimation of correct latency of ERP wave peaks evoked by stimulus. Simultaneous coupling of stimulus event and EEG response onset must be achieved for accurate estimation of 30 the latencies of ERP peaks and event-related oscillations. If there are delays between optically entered stimulus event signal and certain delayed arrival of EEG signal due to filtering and other processes, this delay can be calculated and entered as known time period allowing adjustment of the accurate assessment of matched actual EEG response associated with a stimulus. There is a need to maximally adjust correctness of EEG and stimulus event marks.

For recording event-related potential the correct methodology requires set-up that can accurately synchronize both the stimuli presentation event and the EEG recording. The experimental control software that presents the stimuli should send a marker to the EEG recording system for each stimulus presentation. When these even triggers are accurately recorded along with the EEG signal, then EEG epochs of interest can be correctly segmented for further averaging.

Another important limitation of current art is related to absence of time synchronization of the experimental stimuli with event markers and EEG as it is done in a traditional ERP study (Luck, 2014), as some studies instead use the EEG data in with calculated Bluetooth lag and jitter (Krigolson et al., 2017, 2021). There are several impediments to application of wearable devices for accurate ERP recording. In addition to low sampling rate, relatively artifact prone EEG recording with dry sensors there is one more important technical problem associated with compatibility with recordings of traditional stationary EEG/ERP systems. One of the major issues is correct experimental timing and synchronization or EEG responses with stimulus event mark.

During implementation of typical ERP paradigm stimulus event marker is delivered from an experimental system that generates stimuli to EEG recording computer using cable connection to correctly mark occurrence of the stimulus event in EEG recording. This allows correct segmentation of EEG responses epoch to stimulus event with subsequent averaging for obtaining ERP waveforms.

Several studies using portable and wearable EEG devices tried various methods of attempting precise temporal synchronization of event marks and recorded EEG but none of them described successful resolution of this technical issue. One of the main concerns is associated with temporal jitter and timing variation related to Bluetooth transmission used in wearable EEG devices, as Bluetooth has lag around 20 ms with a jitter affecting correct stamp of event on recorded EEG data.

Availability of correct temporal marking of experimental stimuli to recorded EEG data is critical for obtaining accurate latency values in ERP studies (Luck, 2014). Previous studies with portable EEG systems either did not used markers (Krigolson et al., 2017, 2021) or tried different marking methods that were not precisely synchronized (Debener et al., 2012; Vos et al., 2014; Wong et al., 2014).

There are patents directly addressing the issue of timing synchronization of stimulus event and EEG (U.S. Pat. No. 8,391,966B2, US2020/0390357A1) but their described methodologies either is not intended for wearable mobile EEG devices or are not intended for proposed implementation of cognitive tests nor using single trial methodology with event-related EEG oscillations.

Therefore, there remains a need for a new solution for brain function assessment to overcome the limitations stated above. There is a need for wearable devices that provide high sample rate EEG data with synchronized stimulus event and EEG responses for cognitive testing. More specifically what is needed is a wearable system and method allowing to synchronize presentation of stimuli in visual modality delivered through a controller connected with a wearable device and smartphone or tablet or in auditory modality delivered through the headphones with EEG signal recorded with EEG sensors mounted in the wearable device allowing correct timing of stimulation event and EEG recording during each presented stimulus. What is needed in the art is the ability to record not only EEG metrics but also behavioral responses accurately synchronized with EEG data.

What is needed in the art is a wearable system and method for administration of a battery of cognitive tests such flanker test, auditory and visual oddball tests, and other executive functions tests that provide information about such processes as focused and sustained attention, working memory, cortical inhibition, error monitoring and correction functions.

What is still further needed in the art is an improved methodology of detection of EEG responses to stimuli during cognitive tests, including ability to recognize and identify EEG oscillations not only with the averaging methods but with ability to analyze EEG signal in single trial mode using EEG oscillations occurring in theta and gamma bands in response to stimuli and processed using time-frequency analytical methods based on wavelet transformation.

What is further needed in the art is a practical and effective device and method for cognitive brain assessment and record event-related potentials, evoked and induced EEG oscillations, heart rate, heart rate variability changes, and behavioral responses during cognitive tests to evaluate reaction time of motor response in a form of pressing a button on a controller, and accuracy of responses assessed using such metrics as number of percentage of total errors, number of incorrect responses including those related to missed response, omission errors, or pressing incorrect button or pressing button when response was not required, thus committing commission error.

### SUMMARY OF THE INVENTION

The present invention provides a wearable head mounted device, with headphones, that incorporates embedded EEG and other biometric sensors, and adjacent cognitive tests controller unit. Data collected from the sensors provide data patterns that are analyzed during cognitive tests. The biometric data includes but is not limited to: EEG (electroencephalography), heart rate, pulse volume, heart rate variability (HRV), and other physiological measures. The present invention utilizes for biometrics such physiological signals as EEG recorded from locations which may include at Fz, Cz, and Pz and photoplethysmogram (PPG) or pulse oximetry recorded from the ear. Analysis of EEG and physiological biometric data and presentation of stimulation during cognitive tests is processed using a smartphone or tablet, and/or a remotely located computer.

### BRIEF DESCRIPTION OF THE DRAWINGS

The invention will be better understood according to the following detailed description of several embodiments with reference to the attached drawings, in which:

FIG. 1 illustrates one embodiment of a wearable device with headphones for auditory stimulation, with biosensors and biodata processing and monitoring unit and cognitive interface unit with display for visual stimulation and with buttons to record motor responses;

FIG. 2 illustrates paradigm of arrows flanker test with examples of stimuli in cognitive test;

FIG. 3 illustrates error response-locked error-related negativity (ERN) and error-related positivity (Pe) waveforms;

FIG. 4 illustrates single trial ERN and Pe measures processed using time-frequency wavelet-based transformation;

FIG. 5 illustrates frontal event-related potentials (ERP) in response to target and non-target stimuli in visual cognitive test;

FIG. 6 illustrates evoked and induced EEG gamma oscillations in 35-35 Hz range in response to target and non-target stimuli in visual cognitive test processed using time-frequency wavelet transformation;

FIG. 7 illustrates theta and gamma frequency oscillations and their frequency coupling during a cognitive test.

FIG. 8 illustrates an embodiment of the cognitive test interface which contains an embedded photosensor for visual stimuli onset detection and button press timing detection.

A more detailed understanding of the disclosed device and method will be obtained from the following description of the embodiments along with the figures, drawings, and the claims of the present invention.

### DETAILED DESCRIPTION OF THE INVENTION

Each of the examples of the embodiments of the intention are provided by an explanation of the specifics of the invention, and should not be considered as a limitation of the invention. To those skilled in the art, it is understood that modifications can be made in the present invention within the scope or spirit of the apparatus, system, and methods of the present invention. Further, in this invention, the terms such as “person”, and “user”, and “wearer”, and “patient”, and “human”, and “individual” and “subject” are used interchangeably to refer to a person using the said invention. “Treatment” or “stimulation” or “therapy” or “training” or “session” or “assessment” or “test” as used herein, covers the cognitive assessment of the person/user/wearer/patient/human/individual.

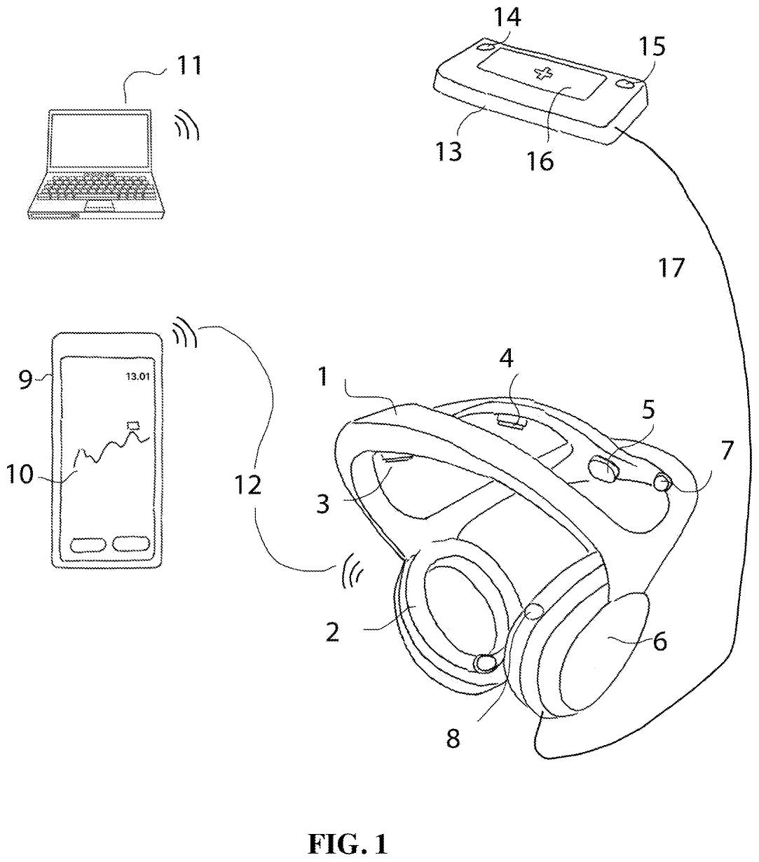

In one aspect presented in FIG. 1, the present invention provides EEG and PPG sensors in a head mounted device 1 with headphones 6 illustrated in FIG. 1. In an embodiment illustrated in FIG. 1, the headphones of the present invention combine EEG (electroencephalography) sensors 3, 4 and 5 for EEG and event-related potential (ERP) and EEG oscillations (ERO) measurement and photoplethysmography (PPG) sensor 8 for heart rate variability (HRV) measurement in a wearable head mounted device with headphones. In an embodiment, a PPG sensor 8 is incorporated inside an over-ear headphone design which reduces ambient noise allowing for increased accuracy. The present invention provides a wearable head mounted headphone set 1 with embedded biometric sensors that collect physiological signals from the user. The device includes Bluetooth (wireless) audio and data transmission 12 which may be used to connect the device 1 to smartphone/mobile device 9, with graphic touchscreen display 10, and said smartphone/mobile device 9 has wireless wi-fi connection with remotely located computer 11. The device 1 may also include a rechargeable battery, speakers, microphone, The wearable device 1 includes a wire 17 connecting headphones with cognitive test interface unit 13.

Electrodes are used to collect EEG signals. FIG. 1 illustrates electrodes that are placed at Fz 3, Cz 4, and Pz 5 locations according to the International 10-20 system and include reference and ground electrodes at A1 or A2 or M1 and M2 locations.

In one embodiment Headphone 6 have built-in microphone 7 for accurate detection of auditory stimuli presentation for timely auditory stimulation timing.

The cognitive test interface unit 13 includes two buttons 14 and 15 for measuring motor responses. The cognitive test interface 13 unit also includes a display 16 for visual stimulation.

The display 16, is made up of a matrix of addressable LEDs, wherein the wearable device 1, is able to update the display 16 via the wire 17.

Photoplethysmography methods are used to collect additional biometrics for measuring heart rate (HR), HRV, and pulse volume. Photoplethysmography (PPG) is an optical measurement of the absorption of specific wavelengths of light by the body. A PPG sensor containing LEDs and photosensors is placed inside one of the earpieces and positioned against the outer ear. The pulse PPG will use a reflectance method for measurement. Placing the pulse sensor 8 inside the earpiece 3 which covers the ear, reduces signal noise from ambient light. The PPG data is converted into the following biometric signals (but not limited to these): heart rate (pulse rate), heart rate variability, and pulse volume.

In the present invention, the user selects the type of cognitive assessment session from a list using their mobile device. The mobile device then wirelessly configures the head mounted device (wearable headset) to execute the cognitive assessment session. Configuration includes the auditory and display information for the different stimuli, stimuli duration, the number of trials to run of each type, and the minimum and maximum duration for each trial. Wherein each different stimuli represents a different trial type. The headset then executes the cognitive assessment session and wirelessly transmits the sensor data and trial metadata to the mobile device. During the test execution the wearable device determines the settings for each trial including randomizing the order, and duration of the trials. The wearable device then executes a fixed interval loop which includes sampling each sensor and button and conditionally triggering updates to the display or playing audio. Wherein the device may utilize the known display update duration and the trial duration settings to precisely control the onset timing of the visual stimuli. The trial metadata includes the sample time, trial type, start or end time for each trial, and the button states and timing.

In one embodiment, said sensor data and test trial metadata is processed by the connected mobile device. Signal processing may include various techniques known to those skilled in the art, including noise filters (i.e., lowpass, highpass, etc.) and analysis techniques (i.e., ERPs, EROs, Fourier transform, Wavelet analysis, etc.). In one embodiment, the wearable headset may execute some or all of the filtering and analysis before transmitting the resulting data to the mobile device. In yet another embodiment the mobile device may execute some or all of the filtering and analysis before transmitting the resulting data to a remotely located server. Wherein the server may perform additional, filtering or analysis. Said analysis may include comparing results to statistical norms or historical data for the user and leveraging machine learning or artificial intelligence.

In the present invention, the cognitive test interface unit includes one or more buttons with timing sensitivity greater than or equal to the sensor sample rate. Wherein the connected wearable headset evaluates the state of said buttons time synchronized to the sampling of the sensors. These buttons provide behavioral motor responses of the user enabling evaluation of reaction time and accuracy, as well as EEG-based assessment of response-locked event-related potentials (ERP) such as ERN and Pe, and error-related EEG oscillations (ERO). The timing of changes to the button states is included in the trial metadata.

The cognitive test interface also consists of a visual display. Said visual display consists of an array of addressable LEDs. Such a display is limited in the shapes and images that can be shown based on the size of the LED array. However, reducing the number of addressable LEDs will reduce the time to redraw the display. The connected headset initiates updates to the display within its sensor sampling loop. As a result, the display built into the cognitive test interface does not run on an independent refresh rate and therefore provides a consistent and known timing delay. Therefore, this display can be considered time synchronized as the connected wearable headset is able to update the display using the same clock timing as its biometric sensors. Wherein the resulting visual stimuli is time synchronized with the biometric sensor data. In this embodiment, the connected headset tracks the display update timing data synchronized with the sensor data and includes the type of visual displayed and the timing information in the trial metadata.

In an alternative embodiment the visual display update timing may utilize a separate clock. In this embodiment, the sensor sampling clock is synchronized to the display clock. Wherein the cognitive test interface unit updates the display timed precisely according to the synchronized clock. Various methods for clock synchronization may be used. In one embodiment the cognitive test interface unit is not wired to the headset. In this embodiment, the cognitive test interface unit utilizes a wireless communication protocol to exchange information. Known methods such as Network Time Synchronization (NTP) or Precision Time Protocol (PTP) can be used to provide clock synchronization. In this wireless embodiment, a mobile device may also be used to initiate updates to the display. Using synchronized clocks the display update timing may be synchronized with the sensor data when the data is processed and analyzed.

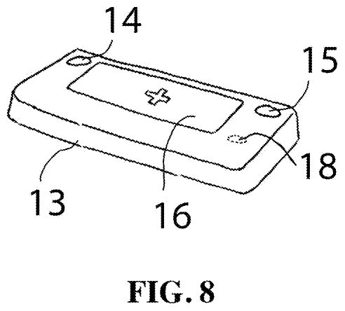

In an embodiment illustrated in FIG. 8, the cognitive test interface unit 13, consists of two buttons 14 and 15, a display 16, and an embedded photosensor 18. The photosensor is placed internally such that 1) external light is blocked from reaching the sensor, 2) light from the display can be directed to the sensor. 3) the outer case covers the area of the display being detected by the photosensor. In this embodiment the display of the cognitive test interface unit is able to run on an independent refresh rate. Wherein updates are provided to the display memory, and the display redraws at fixed intervals. In this embodiment, display updates may be initiated by either the headset, the cognitive test interface unit, or a wirelessly connected mobile device. The cognitive test interface utilizes the built-in photosensor to detect the timing of the display redraw. In this embodiment, the sensor is placed internally to the cognitive test interface unit and is not visible to the user and the timing information is used to analyze the stimulus-locked event-related potentials (ERP) and EEG oscillations (ERO). Alternate types of sensors could be used in this application including measuring changes to the current draw of the display.

Those skilled in the art will understand that different types of display technologies may be used in the present invention. In yet another embodiment, said graphic display on the cognitive test interface unit may be a virtual reality (VR), augmented reality (AR), or mixed reality (MR) display. In this embodiment the cognitive test interface unit may be combined with the wearable headset.

The described wearable headset includes built in speakers which can be used to provide auditory stimulus events. The headset initiates audio events within its sensor sampling loop. In one embodiment the headset electronics and firmware have a fixed delay from the initialization of audio until the audio event plays from the speakers. In this embodiment, the headset tracks the fixed audio timing data synchronized with the sensor data and includes the type of audio played and the timing information in the trial metadata.

In an alternative embodiment, the headset reads the levels of audio output lines to detect the precise timing of the audio events. Depending on the electronic configuration such as the use of an audio codec this may be achieved by monitoring the levels of the audio data lines such as the I2S or SPI lines, or the speakers lines. In yet another embodiment the headset utilizes the built-in microphone to listen to the audio event timing.

The described cognitive test interface unit and connected wearable headset provide a mobile wearable solution for visual and auditory event based cognitive testing. Wherein all stimuli, sensors, and responses are synchronized to a single sample and refresh rate. The present invention improves on prior art as it eliminates the multiple sources of timing latency in a wearable mobile solution for cognitive assessment. The present invention provides a wearable solution for precisely timed data used for stimulus-locked and response-locked event-related potentials (ERP) and EEG oscillations (ERO).

In one example of a cognitive test protocol the present invention may administer a cognitive test with reaction time and accuracy, as well as ERP and EEG oscillation recording at sites Fz 3, Cz 4, and Pz 5 considered as most popular topographic sites for ERP analysis. Other locations and/or alternate locations may also be selected. This protocol may be implemented with a forced-choice neurocognitive test such as Eriksen flanker test (Eriksen & Eriksen, 1974). Modification of this flanker test uses behavioral motor response such as button press and evaluates reaction time and accuracy, as well as EEG-based assessment of stimulus-locked and response-locked event-related potentials (ERP) and EEG oscillations (ERO).

Flanker tests (Eriksen & Eriksen, 197) with EEG recording is a task aimed at assessment of attention using ERP methods. The flanker test is one of the focused attention tasks usable for evaluation of executive functions that include cognitive processes such as selective attention, response inhibition, performance monitoring, and working memory. In the flanker task, users typically decide which of several stimuli have been presented in the middle of the string is the target to respond while simultaneously ignoring the stimuli that are presented at the left and right of the central stimulus, so called flankers. The flanker tasks in the most popular modification requires spatial selective attention and executive control. In this task, irrelevant flankers must be inhibited in order to respond to a centrally located relevant target stimulus. Incompatible trials with incongruent flankers that are different from the central target stimulus result in slower reaction time and increased number of commission errors. Exercise of attention is required to effectively resolve the interference of flanker stimuli and conflict between competing distracting stimuli and responses during performance in the flanker task.

This test is of especial interest since it can readily produce more commission errors and allows analysis of error processing, monitoring and correcting processes believed to be controlled by the frontal and central cortical areas (Falkenstein et al., 2000; Nieuwenhuis et al., 2001). In the flanker test when commission errors are committed the response-locked ERPs of interest are error-related negativity (ERN) and error-related positivity (Pe). The ERN is a negative-going ERP wave that starts peaking around 50 ms post-error. In one embodiment the ERN is measured during a response inhibition paradigm such as the flanker task, wherein users see a target arrow stimulus within a set of other arrow stimuli flanking on both sides of target arrow showing correct direction to press either left or right button. In some trials of the test the flankers are the same as the target (congruent trials) but in other occasions, the flankers are different from the central target arrow (incongruent trials).

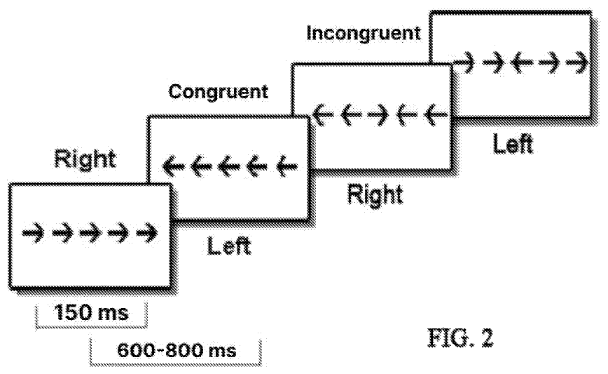

An arrow version of the Eriksen flanker task used widely to elicit the ERN. In some modifications signs like “<” and “>” are used to show direction of response. Example of an arrow flanker test is illustrated in FIG. 2. On each trial, participants view five arrows presented for 150 ms or 200 ms; they are asked to respond as quickly and as accurately as possible to indicate the correct direction of the middle arrow and press the left or right button of the controller. Participants have up to approximately 800 ms-1000 ms from the onset of the stimulus to respond. Half of the trials are congruent (<<<<< or >>>>>), respond left, respond right respectively), whereas the other half are incongruent (e.g., >><>> or <<><<).

Users receive short breaks throughout the task. A longer version of the task can have 360 or even up to 720 trials, while a shorter version of the task is also acceptable but produces less errors. In yet another embodiment a modification of the flanker test is used which includes A No-Go element in the task. The flanker with Go/NoGo task modification combines the flanker task with a Go/NoGo response paradigm (Ruchsow et al., 2005). In this version the visual stimulus includes four arrows and one non-arrow sign such as for example ‘=” presented centrally for 150 ms or 200 ms. The inter-trial interval is 1000 ms. Left-or right-hand responses are required when the middle target arrow is either a “<” (Go press left button) or a “>” (Go press right button). In addition, there are trials with incongruent NoGo stimulus (<<=<< or >>=>>). These NoGo trials (“=” in the center) require responses to be withheld, producing more commission errors. The number of Go congruent, Go incongruent, and NoGo incongruent trials can be adjusted in this flanker protocol modification.

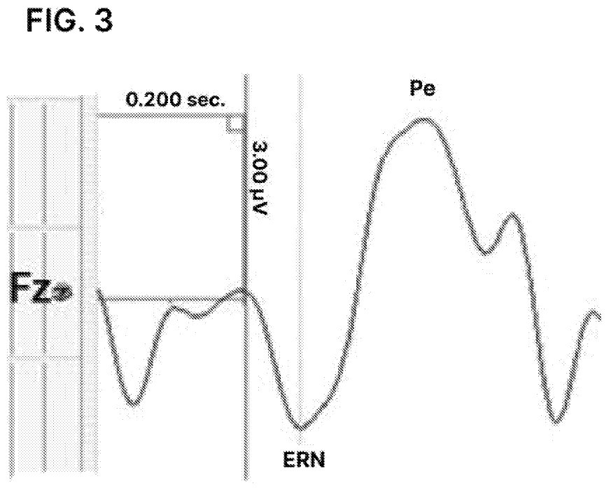

Response-locked ERN and Pe potentials are triggered by committed error response and are reflecting processes related to error detection, error monitoring and those related to error awareness. These error-specific components are the error-related negativity (ERN, more rarely referred to as Ne) and the error-related positivity (Pe). The ERN is a response-locked negative ERP deflection, emerging between 0 and 150 ms after the onset of the incorrect behavioral response-a commission error. The ERN is followed by a positive wave referred to as the Pe potential (100 ms-200 ms range). Waveforms of ERN and Pe stimulus-locked ERP components are illustrated in FIG. 3. The Pe is thought to be related to the conscious recognition of the error or the attribution of motivational significance to the committed error. It is suggested that while the ERN indicates an initial automatic response of error detection, whereas the Pe reflects the conscious comprehension of the error. The ERN/Pe waves are associated with self-monitoring, self-correction and post-error slowing responses, and are interpreted as biomarkers of error processing and committed error awareness (Falkenstein et al., 2000; Nieuwenhuis et al., 2001).

Behavioral response measures in the flanker test may include mean reaction time and response accuracy (percent of correct hits). Number and percent of commission and omission errors are calculated for each test session. Stimulus-locked ERPs in flanker test, including one with NoGo trials in present example of embodiment are posterior (parietal, Pz) N200 ERP and P300 (P3b) in only correct responses to congruent and incongruent stimuli. In flanker test modification with Go-NoGo trials, additional measures of interest include difference wave NoGo-N2 and NoGo-P3 at the Fz site. Both NoGo-N2 and NoGo-P3 are calculated as difference between NoGo-N2 and NoGo-P3 and Go-N2 and Go-P3 at frontal sites (e.g., Fz) within windows typical for N200 (180 ms-320 ms) and P300 (300 ms-500 ms) ERPs. These measures are considered as EEG biomarkers of cortical inhibition processes. They are registered as well in a flanker without NoGo trials.

A longer version of the task can have 360 or even 720 trials, while a shorter version of the task is also acceptable but produces less errors. In embodiments of the flanker test when shorter version is used, and number of error trials is low, the method of analysis of error-related EEG responses in yet another embodiment uses single trial time-frequency analysis based on wavelet transformation.

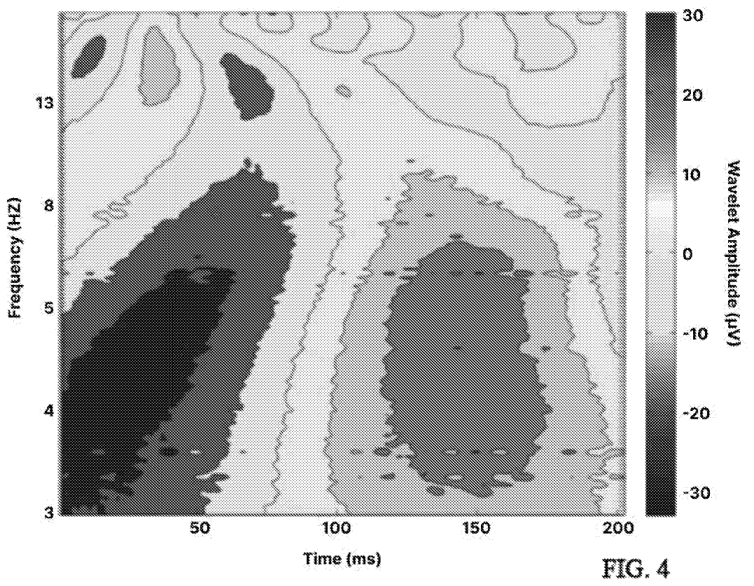

Single-trial analysis of EEG activity is important in order to detect and analyze error-related negativity (ERN) in response to commission error. Method of ERN and Pe analysis using wavelet transformation is suggested to be employed in this embodiment. Single trial EEG data from errors in the flanker task in present embodiment may be processed using a continuous wavelet transform. Coefficients from the transform that corresponded to the theta range are averaged to isolate a theta waveform in the time-frequency domain. Measures called the time-frequency ERN and Pe are obtained from these waveforms for midline frontal and central EEG sites (Fz and Cz) for each error trial. This single trial time-frequency error analysis method is suitable for examining error processing when a user commits only a few errors (Clemans et al., 2012). Illustration of wavelet-based time frequency analysis of single trial ERN and Pe is presented in FIG. 4.

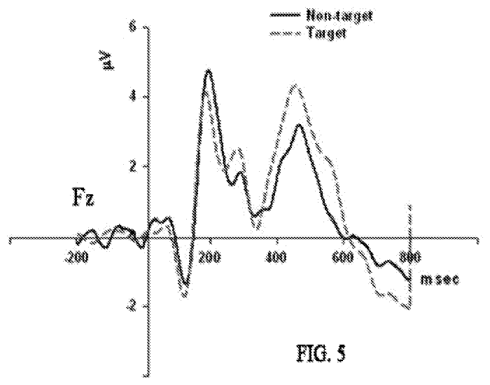

In yet another embodiment of the device and method a visual or auditory oddball test can be used for assessment of cognitive status of the user before or after treatment and used as an outcome measurement tool. In a visual oddball test example, the ERP test paradigm is used for cognitive processes and attention measurement. The visual oddball paradigm is often used to elicit the P3 (P300) cognitive ERP component (Polich & Herbst, 2007; Herrmann & Knight, 2001). In the traditional visual two-stimulus oddball test, the target stimulus is presented infrequently among frequent standard stimuli. In the three-stimulus oddball test version, the rare target stimulus is presented along with frequent standards and infrequently occurring distractor stimuli (might be the same rare stimulus or several novel distractors). The user must respond to the attended target stimulus and ignore any other stimuli. The target and novel stimuli elicit a large positive P3 (or P300) potentials, specifically with the frontal P3a component at Fz and parietal P3b component at Pz electrodes with a peak latency within 300-400 ms post-stimulus. The parietal P3 (P3b) amplitude is interpreted as an update of the mental representation of a stimulus. In the three-stimulus version of visual oddball test novel distracter stimuli elicit frontal P3a ERP, that is interpreted as a marker of attentional orienting. FIG. 5 illustrates P3a component elicited at the frontal Fz site in response to target and non-target stimuli. Therefore, in the three-stimulus modification of visual oddball test with novel distracters, P300 (P3) potential is further divided into the P3a and P3b subcomponents (Polich, 2007). The P3a elicited by an infrequent and uninstructed novel stimulus is localized in the frontal (e.g., Fz) or central (Cz) cortices and has relatively short latency as compared to the P3b component that is elicited in response to attended infrequent stimulus and is localized in the parietal area (e.g., Pz). The P3a component reflects processes related to the selection of stimulus associated with attentional orienting. The amplitude of the P3a reflects processes indicating focal attention. The P3b is reflecting processes related to the allocation of attentional resources during performance in cognitive tasks and is associated with updating working memory. The P3b amplitude is reflecting the attentional resources allocated to processing a stimulus, whereas the P3b latency reflects stimulus classification speed. Target detection in the visual oddball paradigm described above is also associated with a late response at parietal areas (e.g., Pz), starting at 200 ms and continuing for up to 500 ms, including the negative N2 (N200) and positive P3b components. Both P3a and P3b are analyzed to calculate peak amplitude and latency of the peak within preselected window, though in some cases instead of max peak, mean value of component or the area (magnitude is calculated) and in more rare cases N2-to-P3b peak-to-peak amplitude is used. In a most simple modification, two visual stimuli, for example letters “O” and “X” are designed as the standard and target stimuli, respectively. The users are instructed to press “X” for a target stimulus and not to respond for a standard stimulus. Further, the reaction time and correct target detection of the user are recorded. Two types of error are expected: commission error—“false alarm” (i.e., pressed key when standard stimulus was shown, reflects impulsivity) and omission error (forgot to press key when target stimulus appeared, reflects inattention). In yet another example of the embodiment the three-stimulus visual oddball with novel distractors test may be used. This ERP test as stimuli uses letters “X,” “O,” and novel distracters (“v,” “\hat{ }38 , “>,” and “<” signs). One of the stimuli (“O”) is presented on 80% of the trials (frequent standard); the novel stimuli stimulus (e.g., “>”) is presented on 10% (2.5% for each of signs) of the trials (rare distracter), whereas the third (“X”) is presented on the remaining 10% of the trials and represents the target. Users are instructed to press a button when they see the target letter on the screen. Event-related potentials (ERP) locked to stimulus events (triggered by target) reflect the activation of neural structures in primary sensory cortex, and in associative cortical areas related to higher order cognitive processes. The ERP analysis provides temporal information concerning processes such as attention. Earlier ERP components, such as the P100, N100, P200, usually relate to early attentional selection mechanisms, whereas later components (N200, P300/P3b) are more often associated with organization and interpretation of the stimulus. The negative ERP (N200 located over centro-parietal sites occurs within 180 and 320 ms window post-stimulus. This component is believed to be associated with categorization, perceptual closure and attention focusing and signaling about formation of a perceptual representation. The visual N200 is larger if the stimulus contains a perceptual feature or attribute that defines the target to be attended in the test. In a three-stimulus oddball task the P3a is interpreted as orienting response to novel distractions, while the P3b is considered as an index of sustained attention to target.

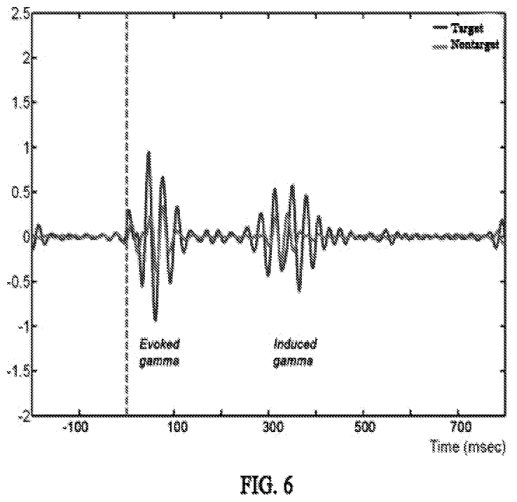

In another embodiment of the device and the methods, the system analysis of EEG responses uses time-frequency wavelet-based analysis of EEG in single trials and more specifically EEG evoked and induced gamma (35-45 Hz or 30-80 Hz) oscillations and EEG frequencies (e.g., theta—4-8 Hz and gamma—35-45 Hz) phase-amplitude coupling methods. In another embodiment of the cognitive tests in present invention wavelet-based time-frequency analysis of event-related EEG gamma oscillations is used. During processing of collected EEG data, the oscillatory response of the 35-45 Hz centered gamma band or wider gamma range (e.g., 30-80 Hz) is broken down into two main groups: evoked and induced responses. These two gamma responses are discriminated based on temporal localization and if they are time-locked to a stimulus. Event-related oscillations are divided into “evoked” and “induced” components depending on their relationship to the event, i.e., stimulus. The early, or so called “evoked” gamma responses occur in the 40-180 ms post-stimulus range. These evoked responses have been attributed to the early information processing linked to the sensory processes and early stages of stimuli perception (Basar, 2013). They are closely time-locked to a specific stimulus. On the other hand, the later occurring induced gamma oscillation responses are better manifested within the post-stimulus window of 250-500 ms range. FIG. 6 illustrates evoked and induced gamma oscillations in 35-45 Hz range waveforms in response to target and non-target stimuli during cognitive test. The induced gamma oscillation responses are observed in tasks requiring higher-order processes of the short-term memory (Herrmann et al., 2014). Event-related gamma oscillations have been associated with indication of perceptual and cognitive processes and considered to be representing an integration of attentional resources and cognitive processes. Previous studies revealed that evoked and induced responses reflect different neural processes and mechanisms (Basar, 2013; Herrmann & Demiralp, 2005; Tallon-Baudry & Bertrand, 1999). It was reported in the art that induced gamma oscillation power is increased at around 40 Hz during a short-term memory task and is related to maintaining an object representation in short-term memory (Tallon-Baudry & Bertrand, 1999). In the present invention, application of single trial evoked and induced gamma oscillations power during cognitive tests such as flanker test or visual or auditory oddball tests with EEG recording are used to assess and evaluate sensory and cognitive processes of the user.

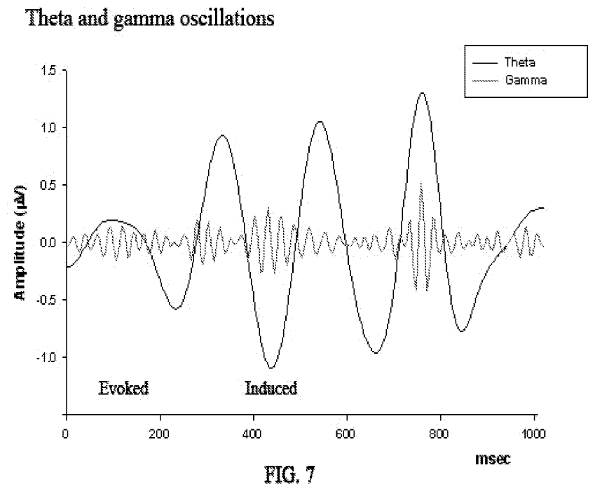

In yet one more embodiment of the present invention the method of cognitive testing examines attentional processes operating in early pre-attentive sensory processes, such as initial orienting, in sustained attention by time-frequency measures of EEG oscillations of several EEG bands during performance on task. Of particular interest in this regard are theta (4-8 Hz) and 40 Hz-centered gamma oscillations. FIG. 7 illustrates phase-amplitude coupling of theta and gamma oscillations during evoked and induced oscillations in response to stimulus in a cognitive test. Previous research suggests that theta oscillations are indicative of neural processes involved in the integration of percepted stimuli in following working memory, attention, and long-term memory processes (Davoudi et al., 2021; Lisman & Jensen, 2013).

In is known to those skilled in the art that EEG oscillations exhibit phase-amplitude coupling in certain physiological states or during performance of specific tasks and research on neural oscillations suggests that the interaction between the brain regions is processed by a cross-frequency coupling between low-frequency band phase and high frequency band amplitude. In particular, the cross-frequency coupling between the theta (4-8 Hz) phase and the gamma (predominantly in 40 Hz centered range, e.g., 35-45 Hz) amplitude may play an important functional role in cognitive activities such as attention and working memory (Canolty et al., 2006). Specifically, EEG responses to visual stimuli are known to be marked by readily observed changes in theta and gamma oscillations. Cross-frequency coupling method in the present method measures the association between the theta oscillation phase and the gamma power. Higher-magnitude theta-gamma coupling values translate into greater gamma amplitude during the theta phase (Lisman & Jensen, 2013). Theta-gamma coupling has been shown to be a functionally important functional role for processes related to short-term and long-term memory. Research suggests that phase-amplitude coupling between the theta phase and gamma amplitudes represents cognitive control mechanisms (Köster et al., 2014). In present embodiment event-related evoked and induced theta and gamma EEG oscillations are analyzed for calculation of theta and gamma activity phase-amplitude coupling for evaluation of cognitive processes of user in response to effects produced by behavioral of any other type of intervention or by self-regulation training using biofeedback or meditation.

In one embodiment, the sensors of the present invention are used to collect biometric signals from the subject during the cognitive assessment including (but not limited to these): EEG (electroencephalography), heart rate, pulse volume, heart rate variability (HRV), and other physiological measures.

One embodiment of the present invention cognitive assessment trials includes the user executing memory based tests. Wherein the user is presented with an image, sound, letters, and or numbers to remember for a set period of time. After another period of time passes during which the user may be distracted by performing another task, the user is asked to identify the remembered item. This embodiment may be combined with analysis of EEG oscillations. In the present invention the measures from cognitive testing may be utilized in the evaluation of cognitive function and or diagnostics of cognitive conditions such as but not limited to mild cognitive impairment (MCI), dementia, Alzheimers, autism spectrum disorder (ASD), obsessive compulsive disorder (OCD), Anxiety, attention deficit disorder (ADD) and attention deficit/hyperactivity disorder (ADHD). Wherein the measures may be compared to statistical norms, calibrated algorithms, or used in artificial intelligence networks.

In another embodiment, said cognitive assessment is performed over the course of one or more treatment sessions and one or more assessment trials.

Alternative embodiments included, but are not in any way limited to, integration of the cognitive assessment modalities of the present invention into other suitable wearable devices besides headphones. One skilled in the art will also understand that application of other known EEG and HRV and ERP and EEG oscillations (ERO) test protocols would be possible within the scope of the current invention. The working examples provided herein are illustrative in nature and are not intended to limit the scope of the disclosure.

The above-described embodiments should be considered as examples of the present invention, rather than as limiting the scope of the invention. In addition to the foregoing embodiments of the invention, review of the detailed description and accompanying drawings will show that there are other embodiments of the present invention. Accordingly, many combinations, permutations, variations, and modifications of the foregoing embodiments of the present invention not set forth explicitly herein will nevertheless fall within the scope of the present invention.

### REFERENCES CITED

### Patent Documents

US 2020/0390357 A1 12/2020 Feiner
U.S. Pat. No. 8,391,966 B2 3/2013 Luo et al
U.S. Pat. No. 8,938,301 B2 1/2015 Hagedorn
U.S. Pat. No. 9,675,292 B2 1/2017 Fadem
EP 2260760 B1 Fadem
US 2012/0330178A1 12/2012 Kraft et al.
US 2019/0216392 A1 7/2019 Bower et al.
US 2021/0338140 A1 11/2021 by Amir
U.S. Pat. No. 9,339,227 B2 5/2016 D′Arcy
U.S. Pat. No. 8,731,650 B2 5/2014 Saida et al. WO2020/223397 A1 11/2020 Lau et al. WO2020/223397 A1 Mcloughlin

### Other Publications

Başar, E. (2013). A review of gamma oscillations in healthy subjects and in cognitive impairment. International Journal of Psychophysiology, 90(2), 99-117. https://doi.org/10.1016/j.ijpsycho.2013.07.005
Canolty, R. T., Edwards, E., Dalal, S. S., Soltani, M., Nagarajan, S. S., Kirsch, H. E., Berger, M. S., Barbaro, N. M., & Knight, R. T. (2006). High gamma power is phase-locked to theta oscillations in human neocortex. Science, 313(5793), 1626-1628. https://doi.org/10.1126/science.1128115
Clemans, Z. A., El-Baz, A. S., Hollifield, M., & Sokhadze, E. M. (2012) Single trial time-frequency domain analysis of error processing in PTSD. Neuroscience Letters. 525(2), 105-110.
Davoudi, S., Ahmadi, A., & Daliri, M. R. (2021). Frequency-amplitude coupling: A new approach for decoding of attended features in covert visual attention task. Neural Computing and Applications. 10.1007/s00521-020-05222-w.
Debener, S., Minow, F., Emkes, R., Gandras, K., & de Vos, M. (2012). How about taking a low-cost, small, and wireless EEG for a walk?. Psychophysiology, 49(11), 1617-1621. https://doi.org/10.1111/j.1469-8986.2012.01471.x
De Vos, M., Gandras, K., & Debener, S. (2014). Towards a truly mobile auditory brain-computer interface: exploring the P300 to take away. International Journal of Psychophysiology 91(1), 46-53. https://doi.org/10.1016/j.ijpsycho.2013.08.010
Eriksen, B. A., & Eriksen, C. W. (1974). Effects of noise letters upon the identification of a target letter in a nonsearch task. Perception & Psychophysics, 16(1), 143-149. https://doi.org/10.3758/BF03203267
Falkenstein, M., Hoormann, J., Christ, S., & Hohnsbein, J. (2000). ERP components on reaction errors and their functional significance: a tutorial. Biological psychology, 51(2-3), 87-107. https://doi.org/10.1016/s0301-0511(99)00031-9
Herrmann, C. S., & Knight, R. T. (2001). Mechanisms of human attention: event related potentials and oscillations. Neuroscience and Biobehavioural Reviews, 25(6), 465-476. http://dx.doi.org/10.1016/S0149-7634(01)00027-6
Herrmann, C. S., Rach, S., Vosskuhl, J., & Strüber, D. (2014). Time-frequency analysis of event-related potentials: A brief tutorial. Brain Topography, 27(4), 438-450. https://doi.org/10.1007/s10548-013-0327-5
Köster, M., Friese, U., Schöne, B., Trujillo-Barreto, N., & Gruber, T. (2014). Theta-gamma coupling during episodic retrieval in the human EEG. Brain Research, 1577, 57-68. https://doi.org/10.1016/j.brainres.2014.06.028
Krigolson, O. E., Hammerstrom, M. R., Abimbola, W., Trska, R., Wright, B. W., Hecker, K. G., & Binsted, G. (2021). Using Muse: Rapid Mobile Assessment of Brain Performance. Frontiers in Neuroscience, 15, 634147. https://doi.org/10.3389/fnins.2021.634147
Krigolson, O. E., Williams, C. C., Norton, A., Hassall, C. D., & Colino, F. L. (2017). Choosing MUSE: Validation of a Low-Cost, Portable EEG System for ERP Research. Frontiers in neuroscience, 11, 109. https://doi.org/10.3389/fnins.2017.00109
Lehrer, P. M., & Gevirtz, R. (2014). Heart rate variability biofeedback: how and why does it work?. Frontiers in psychology, 5, 756. https://doi.org/10.3389/fpsyg.2014.00756
Lisman, J. E., & Jensen, O. (2013). The θγ neural code. Neuron, 77(6), 1002-1016. https://doi.org/10.1016/j.neuron.2013.03.007
Luck, S. J. (2014). Event-related potential technique, 2^{nd} ed. MIT. Nieuwenhuis, S., Ridderinkhof, K. R., Blom, J., Band, G. P., & Kok, A. (2001). Error-related brain potentials are differentially related to awareness of response errors: evidence from an antisaccade task. Psychophysiology, 38(5), 752-760. http://dx.doi.org/10.1111/1469-8986.3850752

Polich, J., & Herbst, K. L. (2000). P300 as a clinical assay: rationale, evaluation, and findings. International journal of psychophysiology, 38(1), 3-19. https://doi.org/10.1016/s0167-8760(00)00127-6

Polich J. (2007). Updating P300: an integrative theory of P3a and P3b. Clinical Neurophysiology 118(10), 2128-2148. https://doi.org/10.1016/j.clinph.2007.04.019
Ruchsow, M., Spitzer, M., Groen, G., Grothe, J., & Kiefer, M. (2005). Error processing and impulsiveness in normals: evidence from event-related potentials. Cognitive Brain Research, 24(2), 317-325. http://dx.doi.org/10.1016/j.cogbrainres.2005.02.003 Salehpour, F., Mahmoudi, J., Kamari, F., Sadigh-Eteghad, S., Rasta, S. H., & Hamblin, M. R. (2018). Brain Photobiomodulation Therapy: a Narrative Review. Molecular Neurobiology, 55(8), 6601-6636. https://doi.org/10.1007/s12035-017-0852-4
Shaffer, F., & Ginsberg, J. P. (2017). An Overview of Heart Rate Variability Metrics and Norms. Frontiers in public health, 5, 258. https://doi.org/10.3389/fpubh.2017.00258
Sherlin, L., Arns, M., Lubar, J., Heinrich, H., Kerson, C., Strehl, U., & Sterman, B. (2011). Neurofeedback and basic learning theory: Implications for research and practice. Journal of Neurotherapy, 15(4), 292-304. https://doi.org/10.1080/10874208.2011.623089
Tallon-Baudry, C., & Bertrand, O. (1999). Oscillatory gamma activity in humans and its role in object representation. Trends in cognitive sciences, 3(4), 151-162. https://doi.org/10.1016/s1364-6613(99)01299-1
Wong, S. W., Chan, R. H., & Mak, J. N. (2014). Spectral modulation of frontal EEG during motor skill acquisition: a mobile EEG study. International journal of psychophysiology, 91(1), 16-21. https://doi.org/10.1016/j.ijpsycho.2013.09.004

What is claimed is:

1. A cognitive test interface system comprising:
a head mounted wearable device incorporating at least one EEG sensor; and
a cognitive test interface unit connected to the wearable device through wired or wireless means;
wherein the at least one EEG sensor is configured to measure biometric data of a wearer of the head mounted wearable device;
and wherein the cognitive test interface unit is configured to administer cognitive testing to the wearer through the provision of visual and/or auditory stimuli.

2. The cognitive test interface system of claim 1, further comprising a mobile device wirelessly connected to one or both of the wearable device or the cognitive test interface unit.

3. The cognitive test interface system of claim 2, further comprising software installed on the mobile device configured to control operation of the at least one sensor on the wearable device and/or the operation of the cognitive test interface unit.

4. The cognitive test interface system of claim 3, wherein the cognitive test interface unit comprises a central controller configured to perform one or more of: administering cognitive testing to the wearer of the head mounted wearable device, receiving biometric data collected by the at least one sensor on the wearable device and interfacing with the mobile device regarding the collection of biometric data or the administration of cognitive testing.

5. The cognitive test interface system of any of claims 2-4, further comprising a remotely located computer connected to the mobile device via the internet or a local area network.

6. The cognitive test interface system of claim 5, wherein the remotely located computer comprises software installed on the remotely located computer configured to provide instructions to, or receive data from, the mobile device.

7. The cognitive test interface system of claim 1, wherein cognitive test data collected upon the administration of cognitive testing is processed to measure EEG, ERP and EEG oscillations.

8. The cognitive test interface system of claim 1, wherein the at least one EEG sensor is placed on the head mounted wearable device according to the international 10-20 system.

9. The cognitive test interface system of claim 1, wherein the head mounted wearable device comprises headphones.

10. The cognitive test interface system of claim 9, wherein the headphones are over the ear type headphones.

11. The cognitive test interface system of claim 1, further comprising at least one additional biometric sensor.

12. The cognitive test interface system of claim 11, wherein the at least one additional biometric sensor is a PPG sensor.

13. The cognitive test interface system of claim 12, wherein the head mounted wearable device comprises over the ear type headphones and the PPG sensor is placed inside the over the ear headphones.

14. The cognitive test interface system of claim 1, wherein the cognitive test interface unit comprises at least one button for measuring motor responses.

15. The cognitive test interface system of claim 1, wherein the cognitive test interface unit further comprises a display for visual stimulation.

16. The cognitive test interface system of claim 15, wherein the display is time synchronized to the EEG sensor and the administered cognitive testing.

17. The cognitive test interface system of claim 15, wherein the display uses an independent clock which is synchronized, in signal processing, to the data from the at least one EEG sensor and cognitive trial test metadata by utilizing an internal sensor built into the display.

18. The cognitive test interface system of any of claims 15-17, wherein the display is selected from the group consisting of a virtual reality display, an augmented reality display and a mixed reality display.

19. The cognitive test interface system of claim 3, wherein the software installed on the mobile device is configured so that a user may select a cognitive testing protocol to be administered to the wearer.

20. The cognitive test interface system of claim 19, wherein upon administration of the selected cognitive testing protocol, biometric data collected by the sensors is displayed on the mobile device or on a remotely located computer.

21. The cognitive test interface system of claim 20, wherein the software on the mobile device or software on the remotely located computer is configured to modulate at least one subsequent round of cognitive testing based upon the biometric data.
